(19) United States
(12) Patent Application Publication
(10) Pub. No.: US 2024/0299728 A1
(43) Pub. Date: Sep. 12, 2024

(54) SYSTEM AND METHOD OF TREATING CARDIOVASCULAR IMPAIRMENT

(71) Applicants: J. Ryan STANFIELD, Sandy, UT (US); James W. LONG, Oklahoma City, OK (US); Michael A. VLADOVICH, Edmond, OK (US); Robert McMANIS, Oklahoma City, OK (US); Kurt DASSE, Oklahoma City, OK (US)

(72) Inventors: J. Ryan STANFIELD, Sandy, UT (US); James W. LONG, Oklahoma City, OK (US); Michael A. VLADOVICH, Edmond, OK (US); Robert McMANIS, Oklahoma City, OK (US); Kurt DASSE, Oklahoma City, OK (US)

(73) Assignee: STAR BP, INC., SPRING, TX (US)

(21) Appl. No.: 18/586,049

(22) Filed: Feb. 23, 2024

Related U.S. Application Data
(60) Provisional application No. 63/489,607, filed on Mar. 10, 2023.

(51) Int. Cl. A61M 60/122; A61M 60/205; A61M 60/508

(52) U.S. Cl. CPC A61M 60/122; A61M 60/205; A61M 60/508

(57) ABSTRACT

A method for implanting a device into a heart of a mammal can include a fluid conduit passing from the atrium into the aorta. The fluid conduit may also include a pump and sensors in the atrium for feedback to a controller for operation of the pump. An outflow portion of the fluid contact may include a diffuser or flow-directing hood for managing type and direction of flow into the aorta.

### CROSS-REFERENCE TO RELATED APPLICATIONS

This application is a nonprovisional application which claims priority from U.S. provisional application No. 63/489,607, filed Mar. 10, 2023, which is incorporated by reference herein in its entirety.

### TECHNICAL FIELD

This disclosure relates to a support system and a method of treatment using a support system.

### BACKGROUND

Heart failure can affect the ability of the heart to provide sufficient blood flow to the organs of the body. Conditions such as coronary artery disease, scar tissue from a myocardial infarction, high blood pressure, and heart valve disease can contribute to congestive heart failure. Pharmaceutical intervention can dilate blood vessels and/or lower blood pressure to allow blood to flow more easily and the heart to pump more efficiently. If the damage to the heart is extensive, surgical intervention or clinical intervention can be required.

### SUMMARY

In one aspect, a method for implanting a device into a heart of a mammal can include inserting a fluid conduit having an inflow portion and an outflow portion into a position in a cardiovascular system of the mammal, wherein the inflow portion is located within an atrium of the heart of the mammal and the outflow portion is located within a portion of an aorta of the cardiovascular system of the mammal, and said fluid conduit passing from the atrium into the aorta.

In another aspect, a system for improving blood flow in a mammal can include a fluid conduit having an inflow portion and an outflow portion, wherein the inflow portion is adapted to be positioned within an atrium of the heart of the mammal and the outflow portion is adapted to be positioned within a portion of an aorta of the cardiovascular system of the mammal, and said fluid conduit passing from the atrium into the aorta.

In certain circumstances, the method can include anchoring the fluid conduit in the atrium so that the outflow portion is downstream into the aorta.

In certain circumstances, the fluid conduit can be configured to be anchored in the atrium so that the outflow portion is downstream into the aorta.

In certain circumstances, the method can include positioning the fluid conduit in the left atrium.

In certain circumstances, the fluid conduit can be configured to be positioned in the left atrium.

In certain circumstances, positioning the fluid conduit in the left atrium can include forming a vector path between a point on the interatrial septum to the descending aorta.

In certain circumstances, the fluid conduit can be configured to be positioned in the left atrium includes forming a vector path between a point on the interatrial septum to the descending aorta.

In certain circumstances, positioning the fluid conduit in the left atrium can include directing the outflow portion downstream into the descending portion of the aorta.

In certain circumstances, the fluid conduit can be configured to be positioned in the left atrium includes directing the outflow portion downstream into the descending portion of the aorta.

In certain circumstances, the fluid conduit can be positioned for optimum washing.

In certain circumstances, the method can include maintaining pressure in the left atrium at an optimum level for a given pathophysiology.

In certain circumstances, the method can include providing a pressure in the left atrium as feedback to a controller for the fluid conduit.

In certain circumstances, the outflow portion can include a diffuser.

In certain circumstances, the outflow portion can include flow-directing hood.

In certain circumstances, the fluid conduit can include a cage to prevent contact with the wall of the left atrium to minimize thrombus formation and ingestion of thrombus into the pump.

In certain circumstances, the fluid conduit can be configured to supply a diffused flow of fluid from the outflow portion.

In certain circumstances, the fluid conduit can be configured to supply a diffused, helical flow of fluid from the outflow portion.

In certain circumstances, the fluid conduit can be configured to supply a flow of fluid from the outflow portion having a velocity of less than 3.0 m/s, less than 2.8 m/s, less than 2.6 m/s, less than 2.4 m/s, less than 2.2 m/s, less than 2.0 m/s, less than 1.8 m/s, less than 1.6 m/s, less than 1.4 m/s less than 1.2 m/s, or less than 1.0 m/s.

In certain circumstances, the fluid conduit can be configured to supply a flow of fluid from the outflow portion having a wall shear stress of less than 4500 dynes/cm², less than 4000 dynes/cm², less than 3500 dynes/cm², less than 3000 dynes/cm², less than 2500 dynes/cm², or less than 2000 dynes/cm².

In certain circumstances, the fluid conduit can be configured to supply a flow of fluid from the outflow portion having a flow rate of less than 10 L/min, less than 8 L/min, less than 6 L/min, less than 4 L/min, less than 3 L/min, less than 2 L/min, or less than 1 L/min.

In certain circumstances, the fluid conduit can be configured to supply a flow of fluid from the outflow portion having a flow rate of greater than 0.30 L/min, greater than 0.40 L/min, greater than 0.50 L/min, greater than 0.60 L/min, greater than 0.70 L/min, or greater than 0.80 L/min.

In certain circumstances, the system can be configured to maintain pressure in the left atrium at an optimum level for a given pathophysiology.

In certain circumstances, the fluid conduit can include a pump.

In certain circumstances, the pump can include a tubular core.

In certain circumstances, the portion of the aorta can be a descending portion of the aorta.

In certain circumstances, the system can include a pressure sensor and a controller for the fluid conduit. The pressure sensor can be configured to provide a pressure in the left atrium as feedback to the controller.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 is a schematic of a support system.

FIGS. 2A-2C are schematics of a pump of a support system.

FIG. 3A-3D are schematics and a sectional view of a pump of a support system as viewed from various sides.

FIG. 4 is a schematic of a pump position in a support system.

FIG. 5 is a schematic of a pump positioned in an atrium.

FIG. 6 is a schematic showing helical flow from a pump into a downstream portion of an aorta.

FIG. 7 is a schematic of a fluid conduit of a support system with sensors.

FIGS. 8A-8B are schematics showing impact of conduit shape on fluid dynamics.

Like reference symbols in the various drawings indicate like elements.

### DETAILED DESCRIPTION

Example configurations will now be described more fully with reference to the accompanying drawings. Example configurations are provided so that this disclosure will be thorough, and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed below could be termed a second element, component, region, layer or section without departing from the teachings of the example configurations.

Systems and methods for assisting circulation through an atrium in the heart of a mammal is described herein. For example, the system and methods for permanently or temporarily improving blood flow performance in an atrium can include assisting blood flow through the left atrium to the aorta. In certain embodiments, a pump can assist with blood flow from the left atrium to the descending portion of the aorta.

Importantly, a percutaneously placed pump can be placed toward the descending aorta rather than the ascending arch of the aorta. In order to achieve therapeutic effect, it is important to minimize damage to the intima of the aorta. In addition, it is important to set the pump design to only operate within a safe window of operation. In doing so, the atrial system can improve quality of life for individuals in various states of heart failure or other compromised cardiac conditions that would benefit from enhancing blood flow from the atrium to the descending portion of the aorta. For example, the pump can be a percutaneous pump, as described herein, or a surgical support pump.

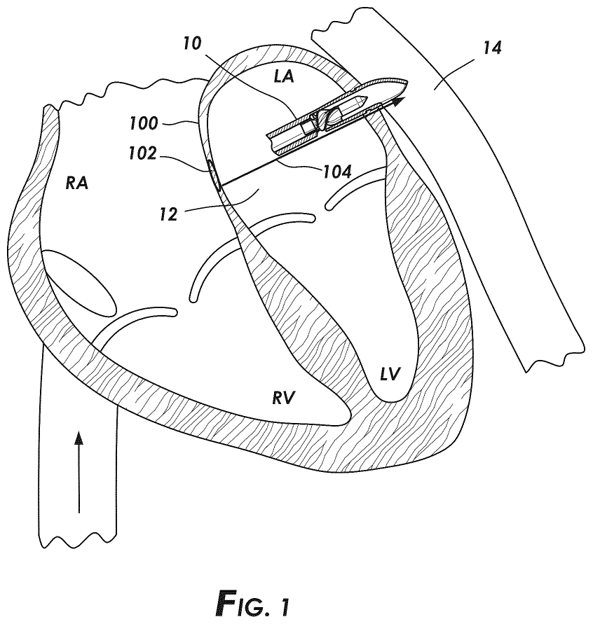

In general, one aspect of the system and method described herein is to provide a support system for improving blood flow in a mammal. The support system can be an atrio-ventricular support system. For example, the support system can decompress the heart and assist systemic blood flow. The support system can include a fluid conduit. The fluid conduit can include a fluid conduit having an inflow portion and an outflow portion. The inflow portion can be adapted to be positioned within an atrium of the heart of the mammal. The outflow portion can be adapted to be positioned within a portion of an aorta of the cardiovascular system of the mammal. For example, the fluid conduit can pass from the atrium into the aorta. Referring to FIG. 1, a fluid conduit 10 can pass from atrium 12 to aorta 14.

Joining of the atrium and the aorta can be achieved with a connector. The connector can have a proximal region, a distal region, and an intermediate region located between the proximal and distal regions. When viewed from either the proximal end or the distal end, the intermediate region of a conduit can define a lumen. The proximal and distal regions can be configured to secure a connector within the cardiovascular system. In some cases, the proximal and distal regions can include a covering. The lumen defined by the intermediate region of the conduit can be supported by a tubular core which can include a septum or valve, for example. The dimensions of a conduit can be adapted to allow a needle or dilator to pass through the intermediate region via the lumen, and then allow the conduit and needle to be inserted into a delivery sheath for placement in the cardiovascular system.

Any appropriate material can be used to construct a connector or regions of a connector. For example, a connector can be constructed as one piece or multiple pieces (e.g., with separate proximal and distal regions). In some cases, a connector can be constructed from compressible, expandable, or malleable materials (e.g., a balloon or shape-memory alloy (nickel titanium (nitinol)). Construction materials can permit the proximal and distal regions of a conduit to be deformed or compressed within the delivery sheath, but regain their original shape (e.g. lip, rim, or disk) when the sheath is retracted. After positioning an expandable conduit, the expandable region can be filled with any suitable material for providing long-term stability (e.g., a polymer capable of crosslinking, thermosetting, or hardening). In some cases, a connector can be constructed from magnetic or paramagnetic materials (e.g., to secure two close, but separate compartments of the cardiovascular system). Regions that are constructed from woven nitinol can be covered in a material for an atraumatic, fluid-tight seal (e.g., biocompatible polymers or fabrics). A tubular core can be constructed from any material that will support the intermediate region (e.g., polymer and/or metal). A septum or valve within a tubular core can be configured to prevent or control blood flow. In some cases, a septum can be adapted to be punctured to permit blood flow (e.g., upon pump placement). Examples of a connector are described, for example, in U.S. Pat. No. 10,137,229, which is incorporated by reference in its entirety.

In certain embodiments, the conduit can include the connector. The conduit can be constructed of expandable or malleable material. The malleable material can be nitinol. The fluid conduit can have a proximal region and a distal region, each of which can be independently adjustable.

In certain embodiments, the fluid conduit can include an intermediate region. The intermediate region can include a pump having a tubular core. The tubular core of the conduit can include a septum or valve.

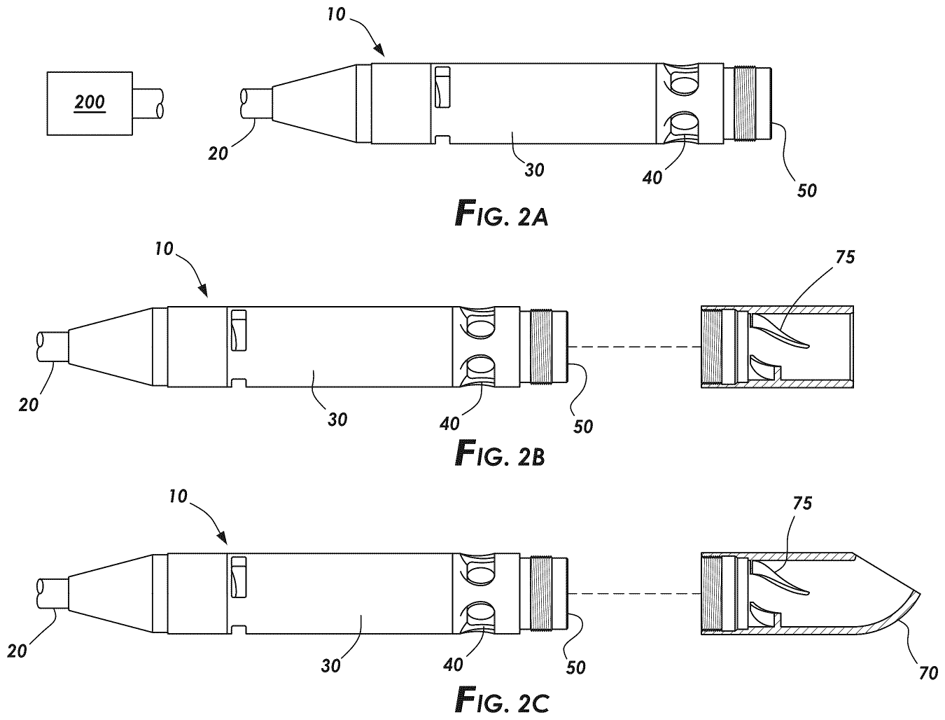

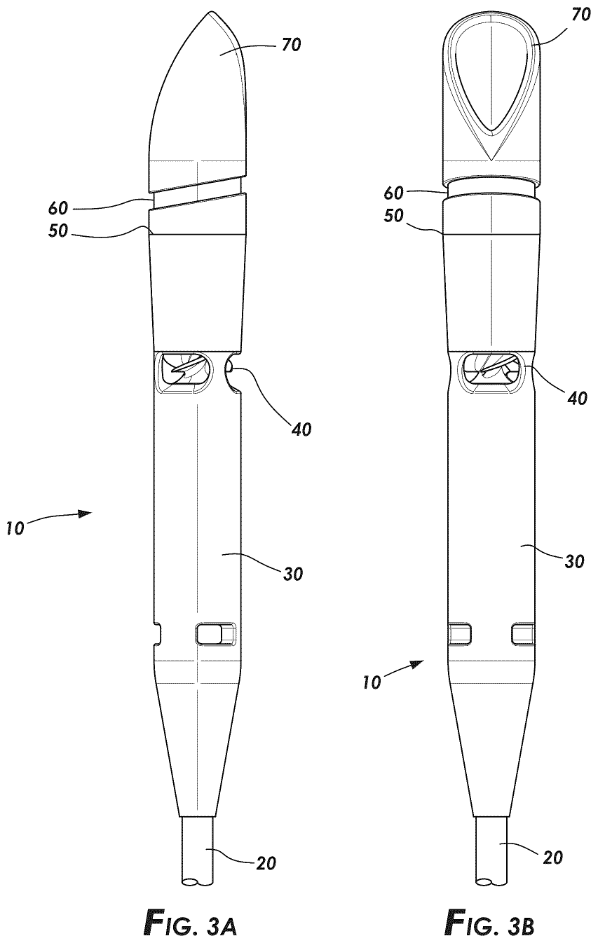

In certain embodiments, the fluid conduit can include a pump. Referring to FIGS. 2A-C and 3A-D, fluid conduit 10 can include a pump having a control line 20 connected to body 30 and a controller 200, which is shown by example in FIG. 2A and also referenced in describing its functions elsewhere herein. The control line 20 may be a percutaneous wire from an externally worn motor controller and rechargeable battery system (collectively forming the controller 200 for some embodiments). In some embodiments, the control line 20 is coupled from the external system components to the pump via the subclavian artery. Alternatively, implantable battery and controller (collectively forming the controller 200) for some embodiments may be used, which is powered via transcutaneous electron transfer (TET).

The body 30 can be a tubular core. Body 30 can include one or more inflow portions 40. The pump can have an outflow portion 50. Referring to FIGS. 2A-C and 3A-D, fluid conduit 10 passes from the atrium to the aorta at tissue interface 60. Outflow of fluid conduit 10 can include a diffuser 75 and/or flow-directing hood 70.

The diffuser 75 can be a fitting from the outflow portion 50 that disrupts the flow of fluid from the outflow portion 50. In some embodiments, the diffuser 75 is a fixed vane within a flow path of blood passing through and exiting the outflow portion 50. The disruption caused by the diffuser 75 may create turbulence and non-laminar flow in the blood and may be shaped to generate/maintain helical flow.

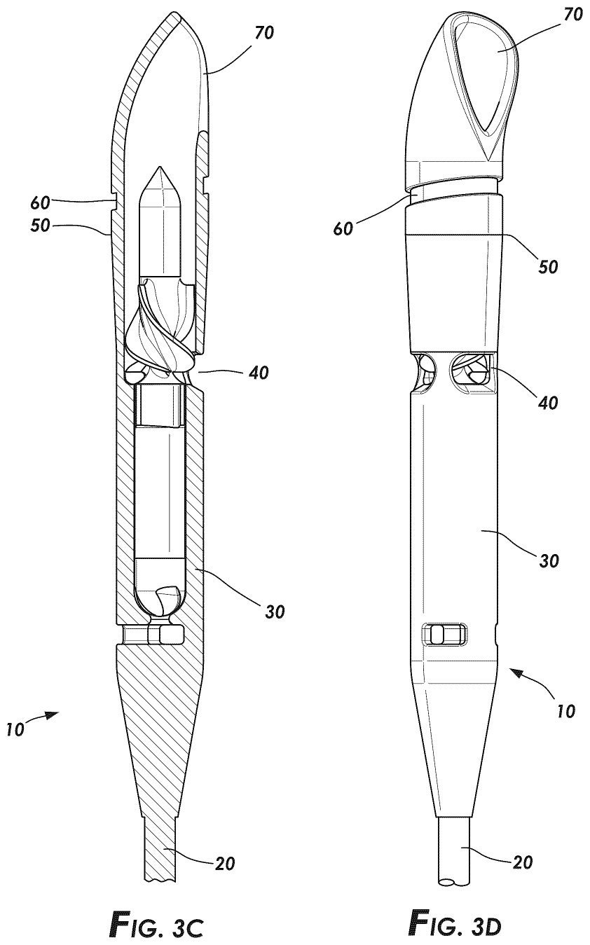

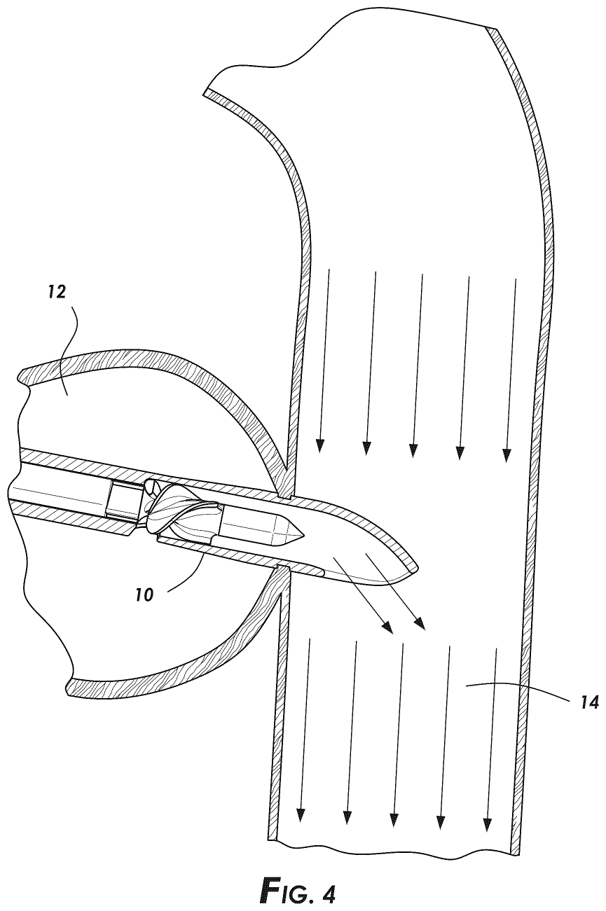

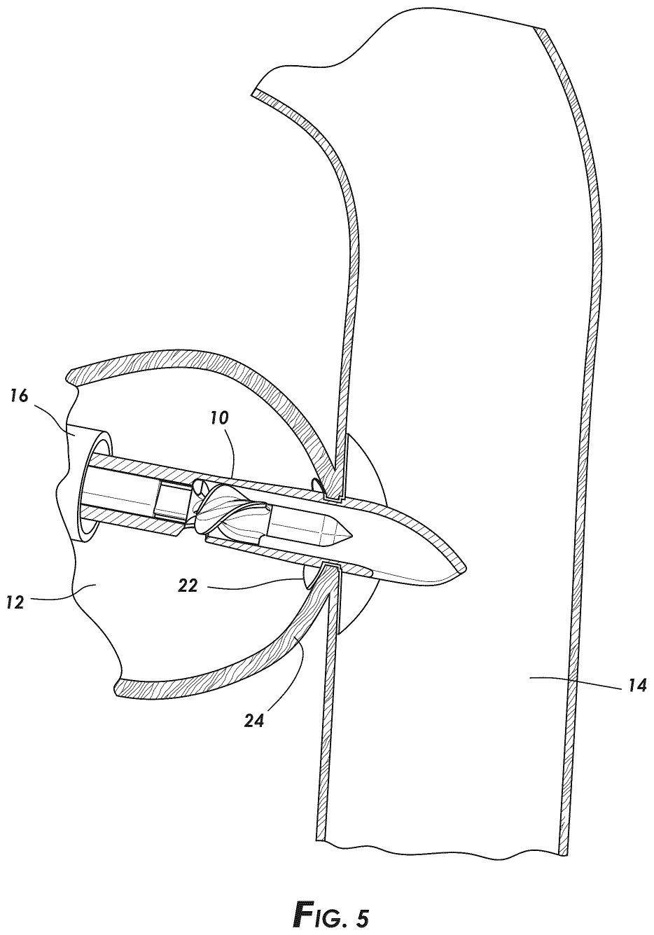

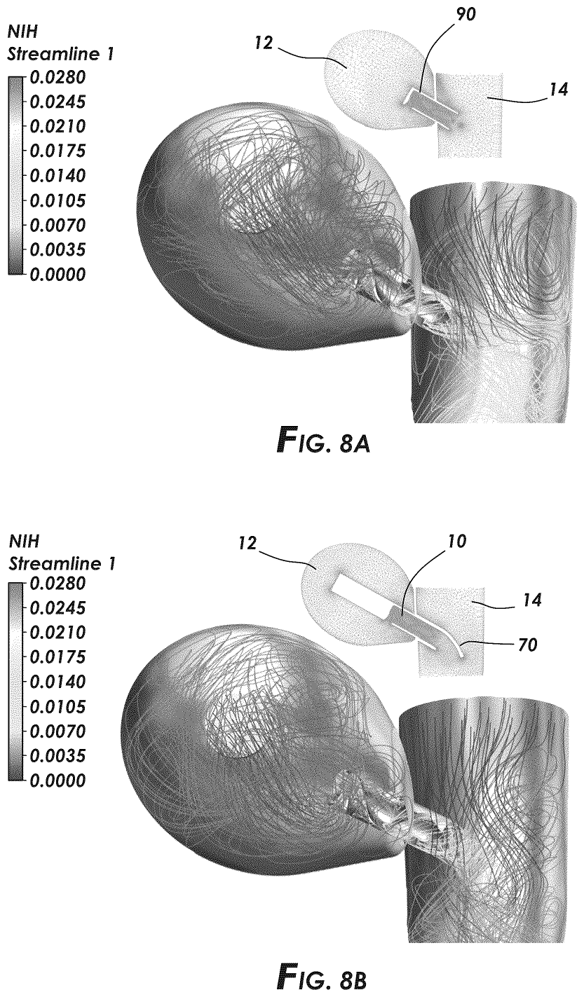

In some embodiments, the flow-directing hood 70 changes axial longitudinal flow output from the outflow portion 50 aligned with a longitudinal axis of the body 30 by an angle (i.e., greater than zero degrees and less than ninety degrees) relative to the longitudinal axis of the body 30. The flow-directing hood 70 may include an extending side directed toward a center of the longitudinal axis of the body 30 to extend in the longitudinal direction over and past an opposing side of the flow-directing hood 70. The resulting orifice of the flow-directing hood 70 thereby may have a non-circular or tear drop shape as shown in FIGS. 3B and 3D and is not formed in a transverse plane to the longitudinal axis at terminus of the body 30. A flow-directing hood, as shown, can thus redirect the flow from the outflow portion to reduce impact of the flow on the tissue of the aorta. In FIG. 8B, flow from the fluid conduit 10 with the flow-directing hood 70 (e.g., as shown in FIGS. 3A-3D) has a lower velocity than an angled and open tip cannula 90 in FIG. 8A (i.e., has a circular shaped outlet formed in a transverse plane to the cannula longitudinal axis at its terminus). The flow-directing hood or diffuser can cause the fluid flow pattern to be directed downstream into the descending portion of the aorta.

In certain embodiments, the fluid conduit can be configured to be positioned in the left atrium, as shown in the example of FIG. 1.

In certain embodiments, the portion of the aorta can be a descending portion of the aorta.

In certain embodiments as shown in FIG. 1, the fluid conduit 10 can be disposed along a vector path 104 between the fossa ovalis 102 on the interatrial septum 100 to where the aorta 14 is descending and is passing in closest proximity to outside of the left atrium 12. The point on the interatrial septum 100 used for defining the vector path 104 origination can be located at or near the fossa ovalis 102. The fluid conduit 10 can be oriented to follow the vector path 104 within atrium 12 and then be directed downstream into the descending portion of the aorta 14. The vector path 104 can be a limit of the angular orientation of the fluid conduit 10 to a predefined vector range (for example, up to a 5 degree variance, 8 degree variance, or 10 degree variance from an ideal orientation defined by the vector path 104) as the fluid conduit 10 courses along the predefined vector range to the aorta 14 that is descending. The orientation of the outflow portion of the fluid conduit serves to control the direction and nature of flow that is most tolerable for patient safety and/or hemocompatibility and/or for minimizing damage to the interior surface of the aorta. This location and orientation can reduce complicating physiologic factors, including thrombus formation.

In certain circumstances, the system and method protect the aorta wall. In certain circumstances, the system can include a structure to reduce physical contact with the aorta wall. In certain circumstances, the method can include creating a fluid flow pattern that reduces damage to the aorta wall.

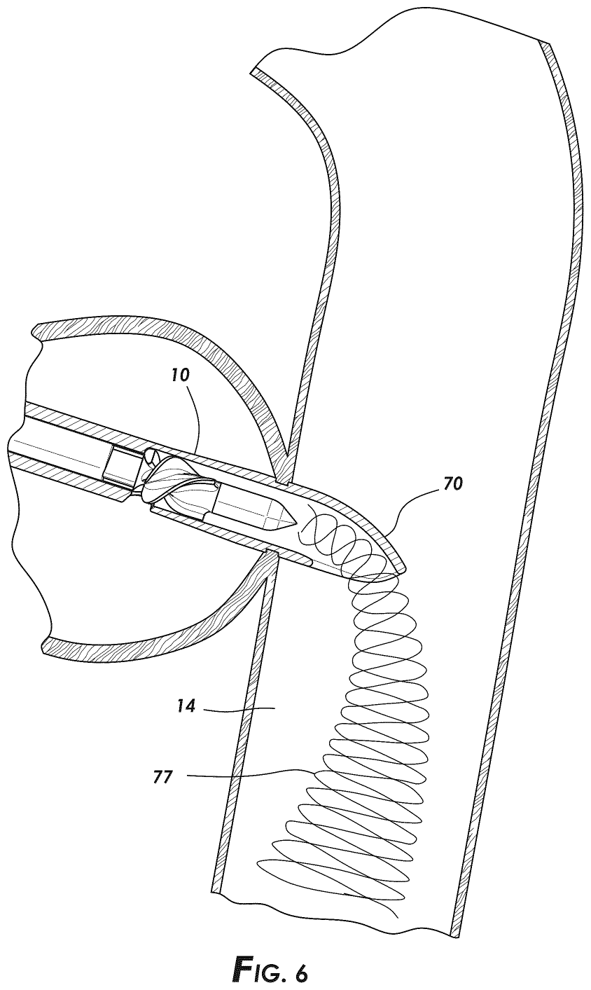

In certain embodiments, the fluid conduit can be configured to be anchored in the atrium so that the outflow portion is downstream into the aorta. For example, as shown in FIG. 5, fluid conduit 10 can be secured by a connector 22 in wall 24 of atrium 12 such that fluid conduit 10 extends into the downstream portion of aorta 14. Fluid conduit 10 can include cage 16, which can position the fluid conduit 10 in the atrium 12 and prevent contact with the wall 24 of the atrium to prevent occlusion, to minimize thrombus formation, and/or to prevent ingestion of a thrombus into a pump. The cage 16 may define an open structure, e.g. shaped wire and/or mesh, having a larger diameter than an outer diameter of the fluid conduit 10.

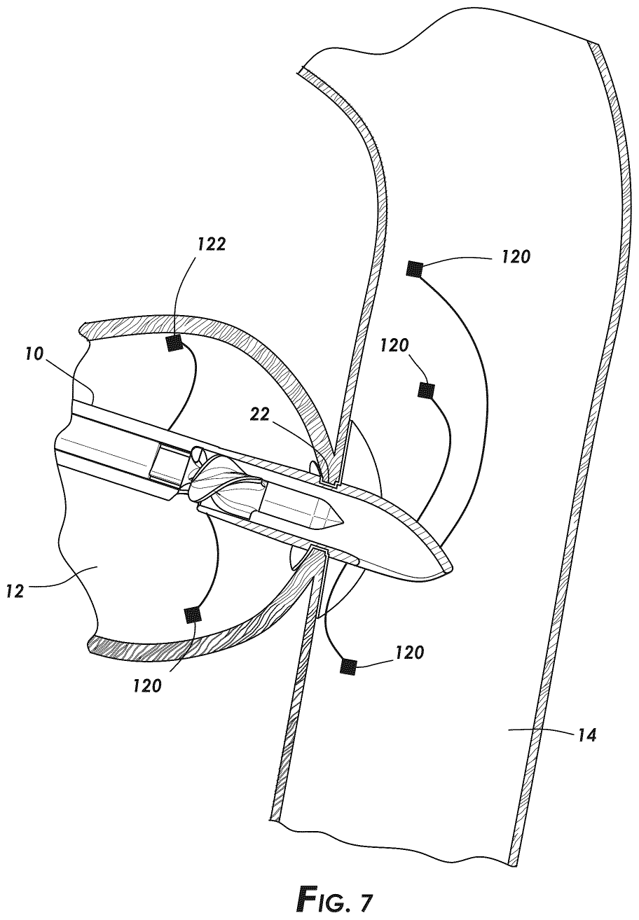

The fluid conduit can provide a diffused outflow of fluid. For example, the fluid conduit 10 can be configured to supply a diffused, helical flow of fluid from the flow-directing hood 70 of the fluid conduit 10. Referring to FIGS. 6, helical flow 77 from a pump into a downstream portion of an aorta 14 can impact the performance of the system. For example, the helical flow 77 can be clockwise with respect to the direction of fluid flow. Alternatively, the helical flow 77 can be counterclockwise with respect to the direction of fluid flow. Other non-laminar flow patterns can be beneficial for the performance of the support system and method described herein.

In certain embodiments, the system can include one or more sensors operatively connected with the controller (e.g., 200 shown in FIG. 2A) for the fluid conduit. In certain embodiments, a device can include one or more sensors configured to provide a pressure in the atrium as feedback to the controller. Referring to FIG. 7, fluid conduit 10 passing from atrium 12 to aorta 14 and secured by connector 22 through wall 24 can include sensors 120 and 122. Sensors 120 can be pressure sensors on the surface of fluid conduit 10 or deployed on adjoined sections of tissue. Sensors 122 can be electrode sensors configured to monitor activity of cardiac muscle. Each of the sensors can provide feedback to the controller which in turn can regulate the speed of the pump and other characteristics that will control one or more of the chamber pressure, overall blood flow rate, fluid velocity or combinations thereof. For example, monitoring pressure can reduce the onset of complications arising from the system. Examples of suitable sensors can include pressure sensors, electrical sensors (such as EKG sensors) or dimensional sensors (such as ultrasonic sensors).

Referring to FIGS. 8A-8B, fluid conduit design can have an impact on flow dynamics at each of the inflow portions and outflow portions of the device. In FIG. 8A, a cannula 90 with an open tip (i.e., has a circular shaped outlet formed in a transverse plane to a cannula longitudinal axis at its terminus) results in relative more disruption of flow in the aorta 14 even tending to inhibit or reverse some existing flow in the aorta 14 from progressing. Further, the flow in atrium 12 entering the cannula 90 causes relative more chaotic flow in the atrium 12. By contrast of FIG. 8A to FIG. 8B, using the fluid conduit 10 having the flow-directing hood 70 provides more streamlined mixing with existing flow in the aorta 14, limited impinging of the flow on walls of the aorta 14, and more streamlined flow into the fluid conduit 10 within the atrium 12.

In some cases, the systems and methods described herein can be used as percutaneous assist devices for enhancing blood flow from a failing heart (e.g., a heart with a congested atrium or failing ventricle). In some cases, the methods provided herein can be used to position an assist device within the heart of a mammal (e.g., within the aorta and left atrium of a human heart). Assist devices provided herein can be configured to reduce the risk of thrombosis and conform to the anatomy of a recipient without causing damage to the heart or aorta.

The system and methods can be used to treat various heart conditions. For example, assist devices provided herein can be used to support the function of a heart to treat congestive heart failure (e.g., left, right, and bilateral failure), heart failure with preserved ejection fraction (diastolic heart failure or HFpEF), heart failure with reduced ejection fraction (systolic heart failure or HFrEF), or heart failure-induced cardiac arrhythmias (e.g., tachycardia and fibrillations). In some cases, devices can be combined to provide a complete heart system (e.g., left and right atrial devices), which can be used to supplement a failing heart. In some cases, devices provided herein can be used to support an impaired heart and to sustain circulation in a patient with end-stage heart failure until a donor heart or an artificial heart can be implanted (e.g., as a bridge to transplant).

In certain embodiments, the fluid conduit can be configured to supply a flow of fluid from the outflow portion having a velocity of less than 3.0 m/s, less than 2.8 m/s, less than 2.6 m/s, less than 2.4 m/s, less than 22 m/s, less than 2.0 m/s, less than 1.8 m/s, less than 1.6 m/s, less than 1.4 m/s less than 1.2 m/s, or less than 1.0 m/s. In certain embodiments, the velocity can be greater than 0.01 m/s, greater than 0.05 m/s, greater than 0.1 m/s, greater than 0.2 m/s, greater than 0.3 m/s, or greater than 0.4 m/s For example, the velocity can be between 0.01 m/s and 3.0 m/s, between 0.05 m/s and 2.6 m/s, between 0.1 m/s and 2.4 m/s, between 0.2 m/s and 2.2 m/s, between 0.3 m/s and 2.2 m/s, or between than 0.4 m/s and 2.0 m/s. In certain circumstances, the maximum velocity should be no more than 1.6 m/s.

In certain embodiments, the fluid conduit can be configured to supply a flow of fluid from the outflow portion having a wall shear stress of less than 4500 dynes/cm², less than 4000 dynes/cm², less than 3500 dynes/cm², less than 3000 dynes/cm², less than 2500 dynes/cm², or less than 2000 dynes/cm² Importantly, the wall shear stress should be. maintained below 4500 dynes/cm² to avoid tissue damage.

In certain embodiments, the fluid conduit can be configured to supply a flow of fluid from the outflow portion having a flow rate of less than 10 L/min, less than 8 L/min, less than 6 L/min, less than 4 L/min, less than 3 L/min, less than 2 L/min, or less than 1 L/min. In certain embodiments, the flow rate can be greater than 0.30 L/min, greater than 0.40 L/min, greater than 0.50 L/min, greater than 0.60 L/min, greater than 0.70 L/min, greater than 0.80 L/min, greater than 1 L/min, greater than 2 L/min, greater than 3 L/min, greater than 4 L/min, or greater than 5 L/min. For example, the flow rate can be between 0.5 L/min and 10 L/min, between 1 L/min and 8 L/min, between 2 L/min and 6 L/min, or between 3 L/min and 5 L/min.

In certain embodiments, suitable flow rates for partial support for HFrEF or HFpEF can be between 0.5 L/min and 3 L/min, for example, between 1.0 L/min to 3.0 L/min.

In certain embodiments, suitable flow rates for full support for HFrEF or HFpEF can be between 0.5 L/min and 10 L/min, for example, between 1 L/min to 8 L/min, preferably up to 6 L/min.

In certain embodiments, the method can include providing a pressure measurement or estimate of a pressure estimate in the left atrium as feedback to a controller for the fluid conduit. In certain embodiments, a sensor can provide the pressure feedback to the controller. The controller can maintain pressure in the left atrium at an optimum level for a given pathophysiology. In certain examples, the optimum level can be about 20 mm Hg. In certain embodiments, relieving atrial pressure can improve physical performance of an individual. Adjustments to atrial pressure relief and to the flow rate and other parameters can be made by monitoring the pulmonary capillary wedge pressure, left atrium pressure, pulmonary artery pressure, left ventricular end diastolic pressure, VO₂, six minute walk parameters, or other physical characteristics.

Traditional left ventricle assist devices (LVADs) for HFrEF uses left ventricular apical cannulation to unload a left ventricle (LV), generally dilated. The support system described herein is designed for HFpEF/DHF and does not use apical cannulation. Apical cannulation in the HFpEF patient can be at risk of obstruction of flow due to overall LV cavity size and septal interference. Apical cannulation can also be undesirable because of the small intralumenal volume of the HFpEF LV coupled with a stiff, thickened LV wall LV wall. Alternatively, the support system described herein uses the left atrium to aorta (LA-Ao) approach to actively decompress the LA and reduce the risk of retrograde pulmonary congestion and the onset of right heart failure. Active decompression of the LA can lead to a reduction in the pulmonary capillary wedge pressure (PCWP) and central venous pressure (CVP).

Haemodynamics should be assessed at baseline preoperatively. In an ideal world the PCWP would be monitored via an implanted pressure sensor and routinely checked at each visit to the hospital. Other methods to assess the condition of the patient is to perform exercise testing using an ergonomic cycle while measuring vital signs and the VO_{2.max}. HFpEF patients tend to have a reduced exercise capacity. Active unloading of the LA and forward systemic flow is designed to increase the exercise capacity of the HFpEF patient as noted by a reduced PCWP and an increase in the six-minute walk. Feedback can be provided to a controller by a wearable device, for example a smart watch or other device.

Using the system described herein, the ideal target pressure in the LA can be maintained between 18 and 25 mm Hg, for example, around 20 mm Hg. The system maintains the filling pressure with pressure sensors to balance and actively decompress and prevent retrograde congestion.

The system described herein can monitor the LV filling pressure or a surrogate measurement for filling pressure. In this way, the pump can actively decompress the left atrium and prevent retrograde pulmonary and right ventricle loading through real time or near real time monitoring of the left atrial pressure (LAP), left ventricle pressure (LVP), PCWP and right atrial pressure (RAP) using sensors or other measurement devices.

Importantly, the system described herein can be used to treat HFrEF or HFpEF, and thus, it is a dual-purpose pump.

For example, pressures can be reduced from greater than 20 mm Hg pulmonary artery wedge pressure in a HFrEF individual to less than 20 mm Hg, for example, 10-15 mm Hg.

In another example, the system can be used to maintain a pulmonary artery wedge pressure in a HFpEF individual to between 18 and 25 mm Hg, for example, around 20 mm Hg.

In another example, a pressure sensor or speed control can be adjusted to assure adequate filling of the left ventricle of a subject with HFpEF.

In certain circumstances, the system and method can adjust the atrial pressure.

In certain circumstances, the system and method can reduce the central venous pressure to less than 15 mm Hg.

In certain circumstances, the system and method can reduce the right atrial pressure to less than 12 mm Hg.

In certain embodiments, the fluid conduit can be positioned for optimum washing. For example, native blood flow within the left atrium provides a wash of the fluid conduit. It can be important to maintain the fluid conduit and pump free of any clot or thrombosis. For example, at least a portion of the fluid conduit components can be coated with a non-thrombogenic surface coating, for example, functionalized acrylate polymers, phosphorylcholine (PC), polyethylene glycol (PEG) or polyethylene oxide (PEO). As shown in FIG. 1, appropriate positioning within the atrium can improve the wash flow.

The mammal can be a human.

In certain embodiments, the percutaneous lead can provide control to the pump. The lead can pass through the interatrial septum and be routed through the venous system to exit the body.

A number of implementations have been described. Nevertheless, it will be understood that various modifications may be made without departing from the spirit and scope of the disclosure. Accordingly, other implementations are within the scope of the following claims.

What is claimed is:

1. A method of implanting a device into a heart of a mammal, comprising:
inserting a fluid conduit having an inflow portion and an outflow portion into a cardiovascular system of the mammal, wherein the inflow portion is located within an atrium of the heart of the mammal and the outflow portion is located within an aorta of the cardiovascular system of the mammal, and the fluid conduit passing from the atrium into the aorta.

2. The method of claim 1, wherein the atrium is a left atrium.

3. The method of claim 1, wherein the atrium is a left atrium and the outflow portion is into a descending portion of the aorta.

4. The method of claim 1, wherein the outflow portion includes a diffuser or a flow-directing hood.

5. The method of claim 1, wherein the fluid conduit includes a structure to prevent contact of a body of the fluid conduit with a wall of the atrium to minimize thrombus formation and ingestion of thrombus into the fluid conduit.

6. The method of claim 1, wherein the fluid conduit is configured to supply a helical flow of fluid from the outflow portion.

7. The method of claim 1, wherein the fluid conduit is configured to supply a flow of fluid from the outflow portion having a velocity of less than 3.0 m/s.

8. The method of claim 1, wherein the fluid conduit is configured to supply a flow of fluid from the outflow portion having a wall shear stress of less than 4500 dynes/cm².

9. The method of claim 1, wherein the atrium is a left atrium and further comprising maintaining pressure in the left atrium between 18 and 25 mm Hg to assist the mammal with HFpEF.

10. The method of claim 1, wherein the atrium is a left atrium and further comprising reducing pressure in the left atrium to between 10 and 15 mm Hg to assist the mammal with HFrEF.

11. The method of claim 1, further comprising providing a signal from a sensor in a left atrium indicative of a pressure in the left atrium as feedback to a controller for the fluid conduit.

12. The method of claim 1, wherein the fluid conduit includes a pump.

13. A system for improving blood flow in a mammal, comprising:
a fluid conduit having an atrium inflow portion and an aorta outflow portion;
a pump disposed in the fluid conduit; and
a controller configured to operate the pump for regulating blood flow through the fluid conduit from the atrium to the aorta.

14. The system of claim 13, wherein the atrium inflow portion is in a left atrium and the aorta outflow portion is in a descending portion of the aorta.

15. The system of claim 13, wherein the controller regulates rate of blood flow through the conduit from the atrium to the aorta or fluid velocity through the conduit from the atrium to the aorta.

16. The system of claim 13, wherein the aorta outflow portion includes a diffuser.

17. The system of claim 13, wherein the aorta outflow portion includes a flow-directing hood.

18. The system of claim 13, wherein the fluid conduit includes a cage to prevent contact of a body of the fluid conduit with a wall of the atrium to minimize thrombus formation and ingestion of thrombus into the pump.

19. The system of claim 13, wherein the fluid conduit is configured to supply a helical flow of fluid from the aorta outflow portion.

20. The system of claim 13, further comprising a pressure sensor coupled to the atrium inflow portion, wherein the pressure sensor is configured to provide a pressure measurement in the atrium as feedback to the controller.
